(19) United States
(12) Patent Application Publication
(10) Pub. No.: US 2017/0079928 A1
(43) Pub. Date: Mar. 23, 2017

(54) COPOLYMERS OF FORMULA (I) AND USES

(71) Applicants: LABORATOIRE FRANCAIS DU FRACTIONNEMENT ET DES BIOTECHNOLOGIES, Les Uli (FR); CENTRE NATIONAL DE LA RECHERCHE SCIENTIFIQUE - CNRS, Paris (FR); UNIVERSITE PARIS-SUD, Orsay (FR)

(72) Inventors: Patrick COUVREUR, Villebon Sur Yvette (FR); Julien NICOLAS, Igny (FR); Thomas BLIN, Antony (FR); Abdessatar CHTOUROU, Elancourt (FR); Nicolas GENEVAZ, Le Kremlin-Bicetre (FR)

(21) Appl. No.: 15/312,007

(22) PCT Filed: May 28, 2015
(86) PCT No.: PCT/FR2015/051408

§ 371 Date: Nov. 17, 2016

(30) Foreign Application Priority Data
May 28, 2014 (FR) 14 54838

(51) Int. Cl. A61K 9/50; C08G 69/40; C08G 69/48; A61K 38/48; A61K 38/37

(52) U.S. Cl. CPC A61K 9/5031; A61K 38/4846; A61K 38/37; C12Y 304/21022; C12Y 304/21021; C08G 69/48; C08G 69/40

(57) ABSTRACT

Disclosed is a copolymer of following formula (I): in which:—x is an integer between 10 and 250, preferably between 40 and 120, —y is an integer between 4 and 100, preferably between 10 and 100, preferably between 19 and 60, —z is an integer between 0 and (100−y), preferably equal to 0, —R represents an alkyl radical having 1 to 10 carbon atoms, a phospholipid, a glycosaminoglycan or an affinity ligand, and —R′ represents a hydrogen, the —CH₂—C≡CH group, a —CH₂-1H-1,2,3-triazole group, a —CH₂—CH₂—CH₂—S—R″ group, in which R″ represents an alkyl radical having 1 to 10 carbon atoms, a phospholipid, a glycosaminoglycan, an affinity ligand or an imaging probe, and the uses of same.

The invention relates to a copolymer of formula (I) below:

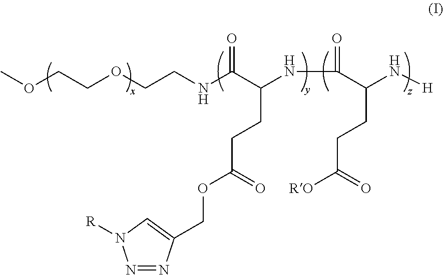

in which:
x is an integer between 10 and 250, preferably between 40 and 120,
y is an integer between 4 and 100, preferably between 10 and 100, preferably between 19 and 60, preferably between 20 and 60,
z is an integer between 0 and (100−y), preferably equal to 0,
R represents an alkyl radical having from 1 to 10 carbon atoms, a phospholipid, a glycosaminoglycan, in particular heparin, or an affinity ligand, and
R′ represents a hydrogen, a —CH₂—C≡CH group, a —CH₂-1H-1,2,3-triazole group, or a —CH₂—CH₂—CH₂—S—R″ group, where R″ represents an alkyl radical having from 1 to 10 carbon atoms, a phospholipid, a glycosaminoglycan, in particular heparin, an affinity ligand or an imaging probe. Preferably, R′ represents a hydrogen.

Biological active ingredients are active ingredients that are widely used in therapy. These active ingredients are sometimes used for treating patients over long periods, sometimes even throughout the patient's life, which involves repeated administrations at a greater or lesser frequency. These repeated administrations often cause considerable drawbacks for the patient to whom the active ingredient is administered.

In general, high doses and a high administration frequency are required to reach and maintain the desired therapeutic or prophylactic effect, which is both restrictive for the patient and expensive.

It has been shown that most biological active ingredients are sensitive to degradation, for example to proteolytic cleavages, which can cause the formation of degradation products devoid of therapeutic or prophylactic effect. This degradation can considerably decrease the half-life and/or the bioavailability of biological active ingredients.

In order to improve the quality of therapeutic and/or prophylactic treatments with biological active ingredients, it would be advantageous to have available pharmaceutical compositions which make it possible to increase the half-life and/or the bioavailability of biological active ingredients, compared with the current pharmaceutical compositions. It would be particularly advantageous to have available pharmaceutical compositions in which the biological active ingredients exhibit improved stability and are less sensitive to degradation, compared with the current compositions.

In particular, it would be advantageous to have available pharmaceutical compositions (or formulations) which make it possible to increase the stability of biological active ingredients in the body and to control their release over time. This would in fact make it possible to decrease the frequency of administration of said formulations, and thus to improve the quality of life of patients and to facilitate the work of practitioners.

An objective of the present invention is to provide means for stabilizing biological active ingredients in formulations administered to a patient, in particular parenterally, and to control their release over time. Such means make it possible in particular to decrease the frequency of administration of said formulations.

A subject of the present invention is thus a copolymer of formula (I) below:

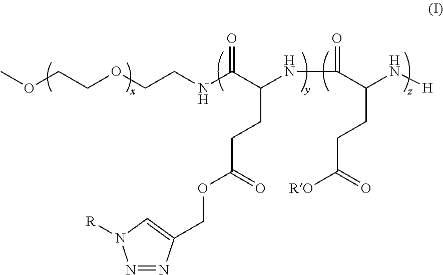

in which:
x is an integer between 10 and 250, preferably between 40 and 120,
y is an integer between 4 and 100, preferably between 10 and 100, preferably between 19 and 60, preferably between 20 and 60,
z is an integer between 0 and (100—y), preferably equal to 0,
R represents an alkyl radical having from 1 to 10 carbon atoms, a phospholipid, a glycosaminoglycan, in particular heparin, or an affinity ligand, and
R′ represents a hydrogen, a —CH₂—C≡group, a —CH₂-1H-1,2,3-triazole group, or a —CH₂—CH₂—CH₂—S—R″ group, where R″ represents an alkyl radical having from 1 to 10 carbon atoms, a phospholipid, a glycosaminoglycan, in particular heparin, an affinity ligand or an imaging probe. Preferably, R′ represents a hydrogen.

Preferably, R′ represents the —CH₂—C≡C group. Such a copolymer is referred to as “copolymer according to the invention” in the present application.

Indeed, such copolymers are capable of forming micelles encapsulating biological active ingredients, in particular proteins, preferably therapeutic proteins, in a formulation. These micelles have in particular the effect of stabilizing said biological active ingredients.

The copolymer according to the invention is a block copolymer. It is composed of two types of blocks:
a block of ethylene glycol monomers, the set of said monomers forming a poly(ethylene glycol) (PEG) block, the first ethylene glycol monomer of which is bonded to a methoxy group,
a block of triazole glutamate-derived monomers, and
optionally glutamate monomers or thioethereal glutamate-derived monomers. In the case where R′ is a —CH₂—CH₂—CH₂—S—R″ group, the term thioethereal glutamate-derived monomers is used. In the case where R′ represents a hydrogen, the term glutamate monomers is used.

More particularly, the PEG block is composed of x ethylene glycol monomers, x being an integer between 10 and 250, preferably between 40 and 120, in particular equal to 45 or 114. The PEG block composed of 45 ethylene glycol monomers has a total molecular weight of 2000 g/mol, and that of 114 ethylene glycol monomers has a total molecular weight of 5000 g/mol.

The copolymer according to the invention comprises y triazole glutamate-derived monomers, y being an integer between 4 and 100, preferably between 10 and 100, preferably between 19 and 60, preferably between 20 and 60, y preferably being equal to 19 or 21. Finally, the copolymer according to the invention may comprise z glutamate monomers or thioethereal glutamate-derived monomers, z being an integer between 0 and (100-y). In one particular embodiment, z is equal to 0.

The term “alkyl” denotes a linear, cyclic or branched hydrocarbon-based radical comprising from 1 to 10 carbon atoms. The alkyl radical having from 1 to 10 carbon atoms is in particular chosen from methyl, ethyl, n-propyl, isopropyl, n-butyl, sec-butyl, isobutyl, tert-butyl, n-pentyl, isopentyl, neopentyl, n-hexyl, n-nonyl, 2-methylcyclopentyl and 1-cyclohexylethyl radicals. Preferably, the alkyl radical has from 4 to 9 carbon atoms and is chosen from n-butyl, sec-butyl, isobutyl, tert-butyl, n-pentyl, isopentyl, neopentyl, n-hexyl, n-nonyl, 2-methylcyclopentyl and 1-cyclohexylethyl radicals.

The term “phospholipid” denotes an amphiphilic lipid, i.e. a lipid consisting of a hydrophilic polar “head” and of two hydrophobic aliphatic “tails”.

Preferably, the phospholipid is chosen from:
phosphoglycerides, the head of which consists of a glycerol 3-phosphate residue esterified with a polar molecule, and the two tails of which are aliphatic chains of two fatty acids; and
sphingomyelins, consisting of sphingosine, of a fatty acid, of a phosphate and of a nitrogenous alcohol.

The phosphoglyceride is preferably chosen from phosphatidic acid, phosphatidylcholine, phosphatidylethanolamine and phosphatidylserine.

The term “glycosaminoglycans” denotes linear poly saccharide polymers, of the type disaccharide formed from a hexose bonded to a hexosamine. Glycosaminoglycans are important constituents of connective tissue extracellular matrices. Among the glycosaminoglycans, mention may preferably be made of chondroitin sulfate, dermatan sulfate, keratan sulfate, hyaluronic acid, heparan sulfate and heparin.

The term “heparin” denotes natural heparin and low-molecular-weight heparins (LMWHs).

Natural heparin is a mixture of various polymers consisting essentially of the following trisulfated disaccharide units: L-iduronic acid 2-O-sulfate and D-glucosamine-N-sulfate, 6-O-sulfate. Heparin is a glycosaminoglycan.

LMWHs are complex sulfonated and glycosylated polymers, produced by chemical or enzymatic depolymerization of heparin. Preferably, the LMWH is chosen from enoxaparin, tinzaparin, nadroparin and fondaparinux (synthetic heparin derivative).

The term “affinity ligand” is intended to mean a molecule which binds reversibly and specifically, via non-covalent interactions (for example electrostatic or hydrophobic interactions, or via hydrogen bonds) with the biological active ingredient, in particular a therapeutic protein.

As affinity ligands, mention may in particular be made of:
substrates or effectors, when the therapeutic proteins are the corresponding enzymes;
antigens, when the therapeutic proteins are the antibodies targeting them; or else
molecules which bind to a receptor, when the therapeutic proteins are said receptors.

Preferably, the affinity ligand is covalently bonded to the copolymer of formula (I) by a group which does not bond to the biological active ingredient.

The term “imaging probe” is intended to mean a molecule which allows the visualization of information for medical purposes and which is used in an imaging technique chosen from fluorescence, X-rays, nuclear magnetic resonance, ultrasound wave reflection, radio-activity, spectroscopy in the near infrared or UV-visible regions and positron emission tomography. As imaging probe, mention may in particular be made of the

FluoroProbe® 547H probe, which emits in the visible region, or the FluoroProbe® 682 probe, which emits in the near infrared region.

The R radical present in the block copolymer of formula (I) may be identical or different among the triazole glutamate-derived monomers, said monomers being present in a number y.

Thus, either R is identical for these monomers and it has the same definition each time, or R is different for these monomers and, in this case, a copolymer with different triazole glutamate-derived monomers is in the end obtained.

Preferably, the R radical present in the copolymer of formula (I) represents an alkyl radical having from 4 to 9 carbon atoms, a phospholipid or a glycosaminoglycan. More preferentially, R is a phospholipid, preferably phosphatidic acid. Phosphatidic acid is a lipid formed by esterification of two fatty acids, which are preferably saturated, and a phosphoric acid with a glycerol. Preferably, the phosphatidic acid according to the invention is formed by esterification of two fatty acids, which are preferably saturated, having a carbon-based chain of from 3 to 22 carbon atoms, and of a phosphoric acid with a glycerol. The term “fatty acid having a carbon-based chain of from 3 to 22 carbon atoms” is intended to mean preferably a fatty acid chosen from butyric acid, valeric acid, myristic acid, palmitic acid, steric acid and arachidic acid.

More preferentially, the phosphatidic acid according to the invention is formed by esterification of two butyric acids and of a phosphoric acid with a glycerol. Alternatively, more preferentially, the phosphatidic acid according to the invention is formed by esterification of two valeric acids and of a phosphoric acid with a glycerol. Preferably, R is the phosphatidic acid of formula below:

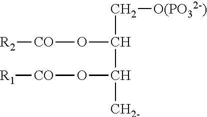

where R₁ and R₂ are identical and represent an aliphatic chain of a fatty acid having from 3 to 22 carbon atoms, preferably R₁ and R₂ are each —(CH₂)₂—CH₃ or —(CH₂)₃—CH₃.

The R′ radical present in the block copolymer of formula (I) may be identical or different among the glutamate or thioethereal glutamate-derived monomers, said monomers being present in a number z.

Thus, either R′ is identical for these monomers and it has the same definition each time, or R′ is different for these monomers and, in this case, a copolymer with glutamate monomers and thioethereal glutamate-derived monomers is in the end obtained.

The subject of the present invention is also the use of a copolymer according to the invention for encapsulating one or more proteins, preferably one or more therapeutic proteins. The therapeutic proteins are in particular chosen from antibodies, coagulation factors (in particular factors I, II, V, VII, VIIa, VIII, IX, X, XI, XII and XIII and from Willebrand factor), modified coagulation factors, factor H, ITI (inter-alpha-trypsin inhibitor), alpha 1-antitrypsin, antithrombin, albumin, fibrinogen, human prothrombin complex, protein C, insulin, interferons and erythropoietins. The term “modified coagulation factor” is intended to mean a fragment of this factor, a protein comprising a fragment of this factor or a mutated factor. Preferably, the therapeutic proteins are modified or unmodified coagulation factors. Preferably, the therapeutic protein is factor VII, preferably in its activated form, FVIII or FIX.

The copolymers according to the invention in fact make it possible to encapsulate biological active ingredients, in particular therapeutic proteins, by the formation of polymeric micelles. These micelles are easily biodegradable, have an average diameter of between 10 and 150 nm, and are injectable, in particular intravenously.

A subject of the present invention is also a pharmaceutical composition comprising, in a pharmaceutically acceptable medium, at least one copolymer of formula (I) according to the invention, and at least one therapeutic protein. The term “pharmaceutically acceptable medium” is intended to mean a medium compatible with administration to a patient.

Preferably, the pharmaceutical composition comprises, in a pharmaceutically acceptable medium, micelles formed by the copolymers of formula (I) according to the invention, said micelles encapsulating the therapeutic proteins.

The pharmaceutical composition preferably comprises a therapeutic protein: copolymer weight ratio of between 1:100 and 100:100.

The pharmaceutical composition is preferably obtained in the following way:
the copolymers are dissolved in water at a concentration of between 5 and 30 mg/ml. The solution is vortexed for 5 to 15 minutes and then passed through an ultrasonic bath for 5 to 30 minutes. The size of the micelles is controlled by light scattering,
the solution of micelles is added to an aqueous solution of therapeutic protein, in a suitable buffer, at a suitable pH,
the mixture is placed between 4 and 25° C., with gentle stirring, for the time required for the encapsulation, i.e. between 5 min and 24 h.

Advantageously, the pharmaceutical composition may contain a stabilizer of amino acid or sugar type.

Advantageously, the pharmaceutical composition may contain an osmotic agent of salt, amino acid or sugar type.

The pharmaceutical composition may be administered in unit administration form. Such suitable unit administration forms include oral forms such as tablets, gel capsules, powders, granules and solutions or suspensions which are oral, sublingual; buccal administration forms such as aerosols, subcutaneous implants; and transdermal, topical, intraperitoneal, intramuscular, parenteral (intravenous, intradermal, intramuscular or subcutaneous), intrathecal, intranasal and rectal administration forms. One preferred form is by injection or infusion, in particular intravenously, in solution or suspension form. Preferably, the pharmaceutical composition according to the invention is suitable for parenteral administration, which includes subcutaneous, intradermal, intramuscular and intravenous administration. Intravenous administration is preferred.

The pharmaceutical composition according to the invention may be in liquid form or in lyophilized form. Preferably, in this case, those skilled in the art understand that, independently of the form in which the pharmaceutical composition is stored (i.e. in lyophilized form or in liquid form), said pharmaceutical composition is administrated to the patient in liquid form.

A subject of the present invention is also micelles obtained from copolymers as defined in the present invention, and comprising an encapsulated therapeutic protein. A subject of the invention is also a pharmaceutical composition comprising the micelles described above.

The subject of the present invention is also a process for preparing a copolymer according to the invention, comprising a step of Click chemistry between the alkyne of formula (II) below:

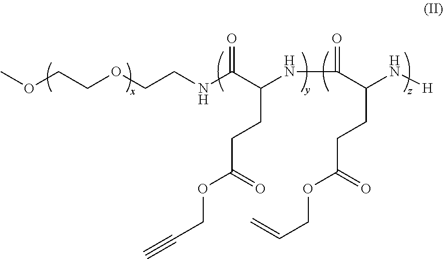

in which:
x is an integer between 10 and 250, preferably between 40 and 120,
y is an integer between 4 and 100, preferably between 10 and 100, preferably between 19 and 60, preferably between 20 and 60,
z is an integer between 0 and (100−y), preferably equal to 0,
   and a compound of formula R—N₃, R being as defined for the copolymer according to the invention, in the presence of copper(I).

The step of Click chemistry between the alkene of formula (II) and the compound R—N₃ makes it possible to obtain the compound of formula (III) below:

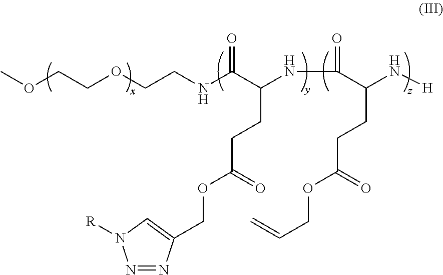

This compound of formula (III) can then:
either undergo a step of oxidation and cleavage of the allyl protective group, in order to obtain a copolymer of formula (I) in which R′ is a hydrogen,
or undergo a step of Click chemistry with a thiol of formula R″—SH, where R″ represents an alkyl radical having from 1 to 10 carbon atoms, a phospholipid, a glycosaminoglycan, an affinity ligand or an imaging probe, in order to obtain a copolymer of formula (I) in which R′ is a —CH₂—CH₂—CH₂—S—R″ group.

Thus, the process for preparing a copolymer according to the invention comprises:

a) a step of Click chemistry between the alkyne of formula (II) below:

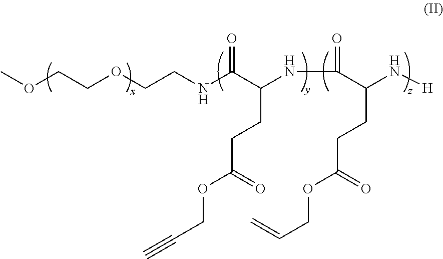

in which:
x is an integer between 10 and 250, preferably between 40 and 120,
y is an integer between 4 and 100, preferably between 10 and 100, preferably between 19 and 60, preferably between 20 and 60, and
z is an integer between 0 and (100−y), preferably equal to 0,
   and a compound of formula R—N₃, R being as defined for the copolymer according to the invention, in the presence of copper(I), in order to obtain the compound of formula (III) below:

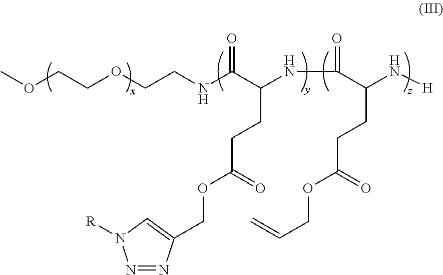

then

b) preferably, when z is other than 0, a step of modifying the allyl function of the compound of formula (III).

According to a first variant, step b) is a step of deprotecting the allyl protective group of the compound of formula (III); preferably, this step comprises oxidation and cleavage of the allyl protective group, in order to obtain a copolymer of formula (I) in which R′ is a hydrogen.

According to a second variant, step b) is a step of Click chemistry between the compound of formula (III) and a thiol of formula R″—SH, where R″ represents an alkyl radical having from 1 to 10 carbon atoms, a phospholipid, a glycosaminoglycan, an affinity ligand or an imaging probe, in order to obtain a copolymer of formula (I) in which R′ is a —CH₂—CH₂—CH₂—S—R″ group.

When z is equal to 0 for the compound of formula (II), step a) of Click chemistry between the alkyne of formula (II) and the compound of formula R—N₃ can also be carried out incompletely, so as to obtain a copolymer of formula (I) according to the invention comprising y triazole glutamate-derived monomers, and z glutamate monomers in which R′ represents a —CH₂—C≡CH group.

A Click chemistry reaction is a simple, high-yield, stereospecific reaction which is carried out without a protective group, and which has a thermodynamic driving force greater than or equal to 20 kcal/mol. Click chemistry reactions make it possible to bond two different units.

The Click chemistry reaction used in the present invention (step a)) involves 1,3-dipolar cycloaddition between an azide function and an alkyne function, so as to form a substituted 1,2,3-triazole. The presence of a copper(I) catalyst makes it possible to obtain exclusively the 1,4-regioisomer, and decreases the reaction time and temperature. Consequently, according to the process of the present invention, the alkyne copolymer of formula (II), i.e. comprising a triple bond, is reacted with an azide, so as to form a copolymer of formula (III) comprising a triazole ring.

Preferably, the copper(I) used in the Click chemistry step according to the invention is present in the form of salts or of complexes.

Such salts are in particular chosen from copper(I) bromide (Cu(I)Br) and copper(I) iodide (Cu(I)I). The complexes are in particular chosen from [Cu(OTf) (C₆H₆)], [Cu (Ph₃P)₃Br] and [Cu(NCCH₃)₄][PF₆].

When copper(I) salts or complexes are used, it is preferable to add, to the medium, an nitrogenous base such as triethylamine, N,N-diisopropylethylamine, PMDETA (N,N,N′,N′,N″-pentamethyldiethylenetriamine), pyridine or 2,6-lutidine.

The Click chemistry reaction is carried out in a medium comprising a solvent. Preferably, the solvent is chosen from toluene, tetrahydrofuran, N,N-dimethylformamide (DMF), dimethyl sulfoxide, acetone, chloroform, acetonitrile, butanol, water, and mixtures thereof.

Preferably, the Click chemistry reaction according to the invention is carried out in the presence of Cu(I)Br, PMDETA and DMF, at a temperature of between 25° C. and 50° C., and for a time of between 15 and 48 hours. More preferentially, the Click chemistry step according to the invention is carried out in the presence of Cu(I)Br, PMDETA and DMF, at a temperature of between 30° C. and 40° C., preferably at approximately 35° C., and for a time of between 20 h and 40 h, preferably of 24 hours.

Preferably, the Click chemistry step according to the invention is carried out, in the presence of copper(I), between the alkyne of formula (II) and the azide derivative of phosphatidic acid (compound of formula R—N₃) of formula (IV) below:

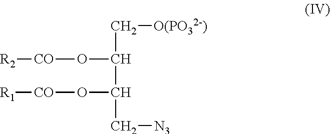

with R₁=R₂=aliphatic chain of a fatty acid having from 3 to 22 carbon atoms, preferably —(CH₂)₂—CH₃ or —(CH₂)₃—CH₃. Preferably, the azide derivative of phosphatidic acid is formed by esterification of two butyric acids and of a phosphoric acid with a glycerol (formula (IV) above with R₁=R₂=—(CH₂)₂—CH₃). Preferably, the azide derivative of phosphatidic acid is formed by esterification of two valeric acids and of a phosphoric acid with a glycerol (formula (IV) above with R₁=R₂=—(CH₂)₃—CH₃).

The azide derivative of formula (IV) used is in particular obtained from diethyl L-tartrate. It makes it possible to obtain in the end a copolymer comprising a phosphatidic acid as R radical.

Preferably, this azide derivative of formula (IV) is obtained by means of the process described in the publication Smith et al., Modular synthesis of biologically active phosphatidic acid probes using Click chemistry, Molecular Biosystems, 2009, 5, 962-972. In particular, it is obtained by means of the process described in scheme 1 of this publication.

The compound of formula R—N₃ may also be azidobutane or azidononane, in order to obtain copolymers of formula (I) in which R is respectively a butyl or nonyl chain.

As described above, the R radical present in the copolymer of formula (I) may be different among the triazole glutamate-derived monomers and the glutamate monomers. In this case, a copolymer with various triazole glutamate-derived monomers and various glutamate monomers is in the end obtained. Such a copolymer of formula (I) can be obtained by means of the following process:

a) by grafting of the molecules having azide-type groups (in particular using Click chemistry) and then

b) by deprotection of the allyl protective group, in order, in the latter case, to regenerate carboxylic acid groups. In this case, a copolymer of formula (I) in which R′ is a hydrogen is obtained.

Preferably, the carboxylic acid functions are deprotected according to the protocol described in the publication by Poché et al., Synthesis of novel γ-alkenyl L-glutamate derivatives containing a terminal C—C double bond to produce polypeptides with pendent unsaturation, Macromolecules, 1997, 30, 8081-8084. The deprotection step can in particular be a step of oxidation and of cleavage of the allyl protective group, in order to obtain a copolymer of formula (I) in which R′ is a hydrogen.

When the carboxylic acid functions protected by the allyl group are not deprotected, this same function can be used in a Click chemistry reaction, termed thiol-ene reaction, with a thiolated derivative of formula R″—SH, or R″ represents an alkyl radical having from 1 to 10 carbon atoms, a phospholipid, a glycosaminoglycan, an affinity ligand or an imaging probe, so as to form the copolymer of formula (V) below:

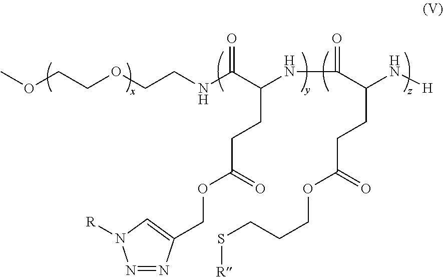

This compound is a copolymer of formula (I), where R′ is a —CH₂—CH₂—CH₂—S—R″ group, with x, y, z, R″ and R as described above.

The invention is now illustrated by various implementation examples.

### EXAMPLES

### Example 1

### Preparation of the m(PEG)₄₅-b-PPLG₂₁ Copolymer

### Step A: Preparation of the PLG-NCA (Propargyl-L-Glutamate-N-CarboxyAnhydride) Monomer

1) Grafting of propargyl alcohol onto glutamic acid:

The reaction is represented schematically as follows:

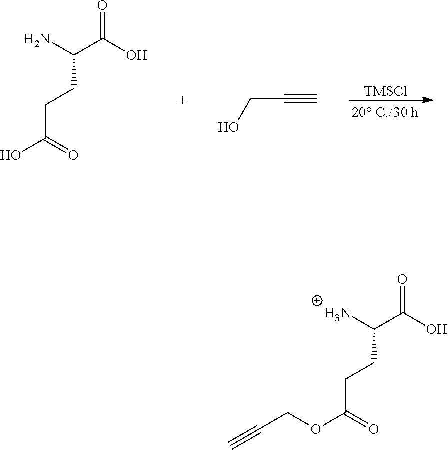

L-Glutamic acid (19.6 g, 133 mmol) and also the propargyl alcohol (500 ml, 8.66 mol) are added to a round-bottomed flask. The solution is then cooled to 0° C. under argon. The trimethylsilyl chloride (36.2 ml, 333 mmol) is then added dropwise to the solution over the course of 1 h. Finally, the solution is mixed at 20° C. for 36 hours. After reaction, the mixture is filtered in order to remove the unreacted product. The product is then precipitated from a volume of diethyl ether corresponding to 10 times the volume of the solution. The precipitate formed is then filtered off, and redissolved in a 10/1 acetonitrile/DMF mixture. After 18 hours at 2° C., the crystals formed are rinsed with cold acetonitrile and finally dried under vacuum. 25.2 g (88% yield) of γ-propargyl-L-glutamate are thus obtained.

2) Closure of the NCA ring, so as to obtain the PLG-NCA monomer:

The reaction is represented schematically as follows:

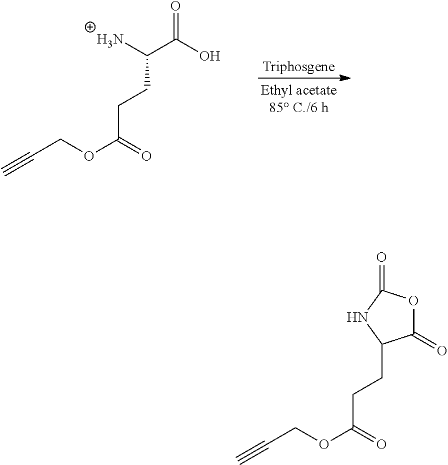

The γ-propargyl-L-glutamate (5.5 g, 24.6 mmol) and the triphosgene (2.4 g, 8.1 mmol) are added to a two-necked round-bottomed flask surmounted by a condenser. The system is then placed under argon while taking care to connect the outlet of the condenser to a solution of KOH (in order to trap any traces of phosgene formed during the reaction). The anhydrous ethyl acetate (150 ml) is then added. The mixture is then placed under argon bubbling at 85° C. for 6 hours. After reaction, the mixture is filtered, then washed very rapidly with 30 ml of cold water and then with 30 ml of a saturated NaCl solution. The organic phase is then dried with MgSO₄, then filtered and concentrated under vacuum. The PLG-NCA product (2.9 g, 55% yield) is stored in a freezer under argon in order to prevent any risk of opening of the N-carboxyanhydride ring.

Step B: Ring-Opening Polymerization of the PLG-NCA Monomer Initiated by mPEG-NH₂, So as to Obtain the m(PEG)₄₅-b-PPLG₂₁ Copolymer

The reaction is represented schematically as follows: mPEG₄₅-b-PPLG₂₁ copolymer (compound of formula (II))

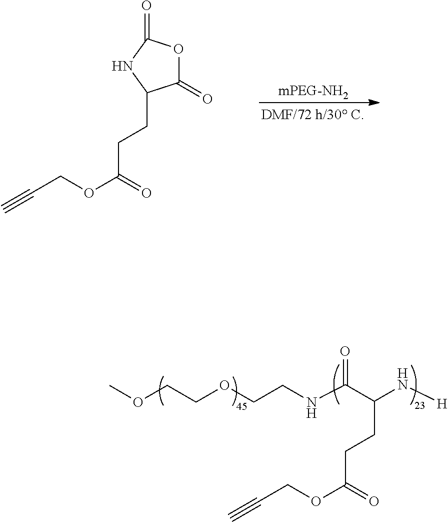

For synthesizing an mPEG₄₅-b-PPLG₂₁ copolymer, the synthesis is described as follows.

The mPEG-NH₂ (0.242 g, 0.121 mmol) is added to a two-necked flask surmounted by a condenser, the end of which is connected to a bubbling system. The system is then placed under an argon system. The anhydrous DMF is then added (15 ml). Finally, a solution of PLG-NCA (0.5068 g, 2.425 mmol) in anhydrous DMF (5 ml) is added to the previous mixture. After 72 hours of reaction at 30° C., the product is simply precipitated from 300 ml of cold diethyl ether, redissolved in 10 ml of DMF and then precipitated once again from a 300 ml volume of cold diethyl ether. The polymer is then filtered and then dried under vacuum overnight.

### Example 2

### Preparations of Copolymers of Formula (I) According to the Invention

1) Copolymers obtained with azidobutane or azido-nonane:

Step A: Preparation of Azidobutane (C4) and of azido-nonane (C9)

The reaction is represented schematically as follows:

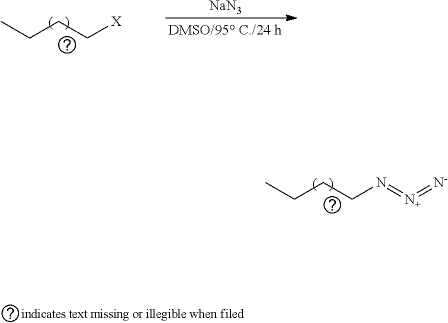

with X═Br or I.

For synthesizing azidobutane, the synthesis is described as follows.

The iodobutane (10 g, 54 mmol) and also the sodium azide (5.26 g, 81 mmol) and the anhydrous dimethyl sulfoxide (100 ml) are added to a round-bottomed flask placed under argon. The system is then placed at 95° C. for 24 hours. After reaction, the solution is cooled and then mixed with a solution of water. The aqueous phase is then extracted with diethyl ether. The organic phase is then washed with water and then dried with MgSO₄. Finally, the diethyl ether is evaporated off by means of a rotary evaporator and the azidobutane (4.3 g, 43 mmol, 80% yield) is dried under vacuum.

Step B: Click Chemistry Reaction Between the m(PEG)₅-b-PPLG₂₁ Copolymer Obtained in Example 1 and Azidobutane, So as to Obtain a Copolymer of Formula (I) According to the Invention (Hereinafter “Copolymer C4”)

The reaction is represented schematically as follows: Copolymer C4.

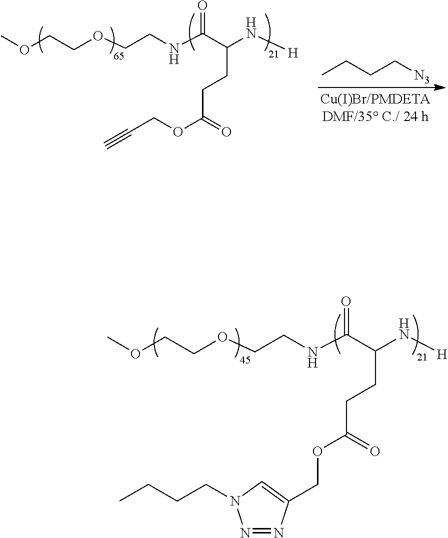

The mPEG₄₅-b-PPLG₂₁ (0.4 g, 1.5 mmol of alkyne function), the azidobutane (0.297 g, 3 mmol) and the dimethylformamide (10 ml) are added to a round-bottomed flask. This mixture is degassed under argon for 30 minutes. Furthermore, a solution of Cu(I)Br (0.1075 g, 0.75 mmol), of PMDETA (313 μl, 1.5 mmol) and of dimethylformamide (5 ml) is degassed for 30 minutes. After degassing, the latter solution is added to the round-bottomed flask and the mixture is stirred for 24 hours at 35° C. The solution is then redissolved in 10 ml of dimethyl sulfoxide and the mixture is then dialyzed for 7 days against a 10 mM EDTA solution and then for 5 days against a milli-Q water solution. Finally, the copolymer C4 (0.4735 g) is recovered after lyophilization.

Step C: Click Chemistry Reaction Between the m(PEG)₄₅-b-PPLG₂₁ Copolymer Obtained in Example 1 and Azidononane, So as to Obtain a Copolymer of Formula (I) According to the Invention (Hereinafter “Copolymer C9”)

The reaction is represented schematically as follows: Copolymer C9.

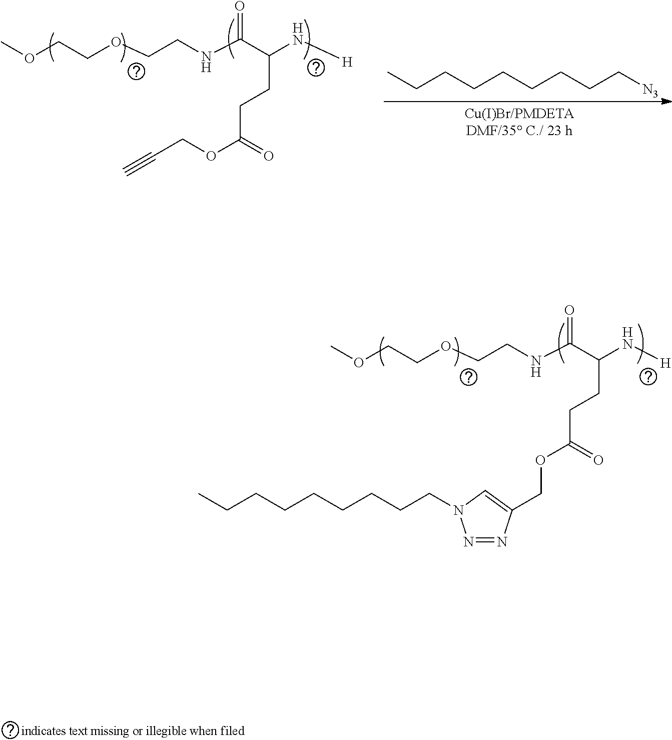

The mPEG₄₅-b-PPLG₂₁ (0.4 g, 1.5 mmol of alkyne function), the azidononane (0.777 g, 3 mmol) and the DMF (10 ml) are added to a round-bottomed flask. This mixture is degassed under argon for 30 minutes. Furthermore, a solution of Cu(I)Br (0.1075 g, 0.75 mmol), of PMDETA (313 μl, 1.5 mmol) and of dimethylformamide (5 ml) is degassed for 30 minutes. After degassing, the latter solution is added to the round-bottomed flask and the mixture is stirred for 24 hours at 35° C. The solution is then redissolved in 10 ml of dimethyl sulfoxide and the mixture is then dialyzed for 7 days against a 10 mM EDTA solution and then for 5 days against milli-Q water. Finally, the copolymer C9 (0.5576 g) is recovered after lyophilization.

2 Copolymers obtained with the azide derivative of phosphatidic acid:

### Step A: Preparation of the Azide Derivative of Phosphatidic Acid (Product A1)

The preparation of the azide derivative of phosphatidic acid requires six synthesis steps.

Step 1: The first step of this synthesis consists in protecting the diol function with an acetal.

The reaction is represented schematically as follows:

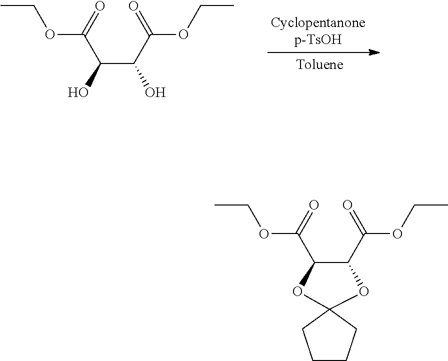 [SMILES: CCOC(=O)[C@H](O)[C@@H](O)C(=O)OCC ; CCOC(=O)[C@@H]1OC2(CCCC2)O[C@H]1C(=O)OCC]

The diethyl L-tartrate (4.05 g, 19.6 mmol) and the toluene (130 ml) are added to a round-bottomed flask. The cyclopentanone (8.7 ml, 98 mmol) and the p-toluene-sulfonic acid (373 mg, 1.96 mmol) are added to this solution. An azeotropic distillation is then carried out at 130° C. for 15 hours by means of a Dean-Stark apparatus. Solid sodium bicarbonate (329 mg) is then added to the solution and the stirring is maintained for a further 10 minutes. The reaction medium is filtered and the filtrate is concentrated by means of a rotary evaporator. Finally, the product A1 (4.5 g, 16.5 mmol, 85% yield) is obtained in the form of a yellow oil after purification on a silica chromatography column (eluent: cyclohexane, retention factor of 0.4, revealer: phosphomolybdic acid) and evaporation.

Step 2: Reduction of the ester groups to alcohol groups

The second step consists in reducing the ester groups to alcohol functions, so as to obtain the product A2. The reaction is represented schematically as follows:

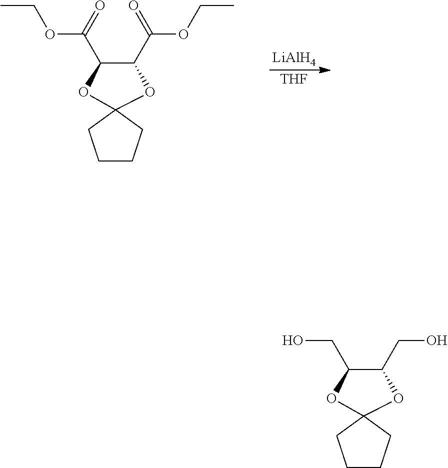 [SMILES: CCOC(=O)[C@@H]1OC2(CCCC2)O[C@H]1C(=O)OCC ; "LiAlH4" ; OC[C@@H]1OC2(CCCC2)O[C@H]1CO ; "THF"]

The product A1 (5 g, 18.4 mmol) is diluted in anhydrous

THF (16 ml) under argon. A solution of LiA1H₄ (1.4 mg, 36.7 mmol) in anhydrous THF (20 ml) is then prepared at 0° C. under argon. The solution of the product Al is added dropwise to the solution of LiAlH₄ at 0° C. Once the addition is complete, the reaction medium is stirred for a further 1 h at 0° C., then for 1 h at ambient temperature. The solution is then cooled to 0° C. Successive and very slow additions of water (2 ml), of 10% NaOH (4 ml) and of water (2 ml) are then carried out in order to stop the reaction. The reaction mixture is stirred for a further 30 minutes and dried over MgSO₄ for 30 minutes. The solution is finally filtered and concentrated by means of a rotary evaporator.

Finally, the product A2 (3.3 g, 17.4 mmol, 95% yield) is obtained in the form of a white solid after purification on a silica chromatography column (eluent: ethyl acetate, retention factor =0.48, revealer: phosphomolybdic acid) and evaporation.

Step 3: Substitution of an alcohol with an azide function

The third step of the synthesis consists in substituting an alcohol function with an azide function, so as to obtain the product A3.

The reaction is represented schematically as follows:

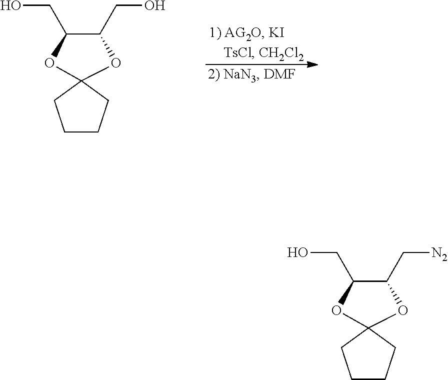

The product A2 (1.44 g, 7.66 mmol) is suspended in dichloromethane (40 ml), in a round-bottomed flask. After complete dissolution, the silver oxide (2.66 g, 11.5 mmol), the tosyl chloride (1.606 g, 8.42 mmol) and the potassium iodide (128 mg, 0.766 mmol) are then added to the suspension. The resulting solution is then stirred at ambient temperature for 2 h. In order to remove the silver oxide, the reaction medium is filtered through a small silica column using ethyl acetate as eluent. The filtrate is concentrated by means of a rotary evaporator. The DMF (80 ml) and the sodium azide (1.244 g, 19.16 mmol) are then added to the filtrate. The solution is then stirred at 85° C. for 15 hours. The solution is concentrated by means of a rotary evaporator. Water (30 ml) is added and then the product is extracted with chloroform (3×100 ml), and the organic phase is dried with MgSO₄ and concentrated by means of a rotary evaporator.

Finally, the product A3 (1.3 g, 6.1 mmol, 80% yield) is obtained in the form of an orangey liquid after purification on a silica chromatography column (eluent: 1/1 v/v ethyl acetate/cyclohexane, retention factor of 0.57, revealer: phosphomolybdic acid) and evaporation.

Step 4: Substitution of the alcohol with a phosphotriester group

The fourth step of the synthesis consists in substituting the remaining alcohol function with a phosphotriester group, so as to obtain the product A4.

The reaction is represented schematically as follows:

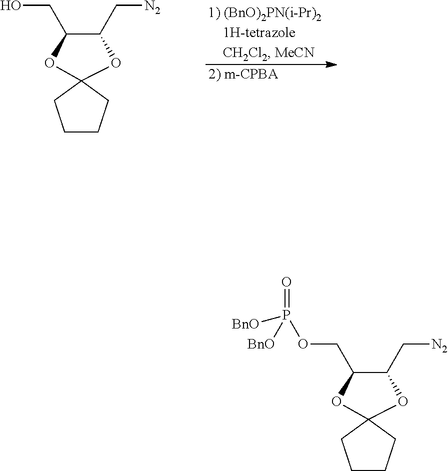

with

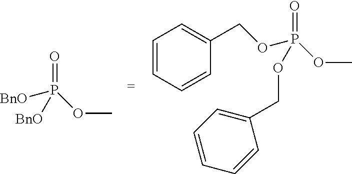 [SMILES: COP(=O)(OCc1ccccc1)OCc1ccccc1 ; COP(=O)(OCc1ccccc1)OCc1ccccc1]

The product A3 (850 mg, 3.98 mmol) is dissolved in 20 ml of anhydrous dichloromethane, in a round-bottomed flask. The 1H-tetrazole (26.6 ml, 11.96 mmol, 0.45 M) is then added and the solution is placed at 0° C. under argon. The dibenzyldiisopropylphosphoramidite (1.442 ml, 4.38 mmol) is then added dropwise. The stirring is continued for 10 minutes at 0° C. and then for 1 h at ambient temperature. At this point, the solution is again cooled to 0° C. and the m-chloroperbenzoic acid (2.06 g, 11.96 mmol, 57% purity) is added to the solution. The stirring is continued for 1 h 30. The reaction is stopped by adding a saturated sodium bicarbonate solution (40 ml). The solution is then extracted with dichloromethane (3×100 ml), dried with MgSO₄ and, finally, concentrated by means of a rotary evaporator.

Finally, the product A4 (1.5 g, 3.1 mmol, 80% yield) is obtained in the form of a pale yellow oil after purification on a silica chromatography column (eluent: 1/1 v/v ethyl acetate/cyclohexane, retention factor of 0.61, revealer: phosphomolybdic acid) and evaporation.

Step 5: Deprotection of the diol function

The fifth step of the synthesis consists in deprotecting the diol function, in order to obtain the product A5.

The reaction is represented schematically as follows:

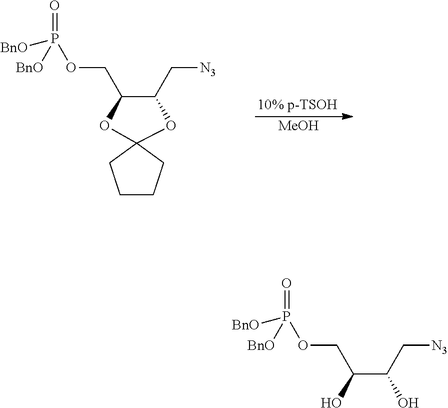 [SMILES: "MeOH" ; [N-]=[N+]=NC[C@@H]1OC2(CCCC2)O[C@H]1COP(=O)(OCc1ccccc1)OCc1ccccc1 ; "10% p-TSOH" ; [N-]=[N+]=NC[C@H](O)[C@@H](O)COP(=O)(OCc1ccccc1)OCc1ccccc1]

The product A4 (1.605 g, 3.39 mmol) is dissolved in the methanol (25 ml), in a round-bottomed flask. The p-toluenesulfonic acid (64.5 mg, 0.339 mmol) is added with stirring. The solution is stirred at ambient temperature for 15 h and the reaction is stopped by adding a saturated sodium bicarbonate solution (30 ml).

The product is then extracted in chloroform (2×100 ml). The organic phase is dried with MgSO₄ and concentrated by means of a rotary evaporator.

Finally, the product A5 (0.873 g, 2.1 mmol, 55% yield) is obtained in the form of a pale yellow oil after purification on a silica chromatography column (eluent: 8/2 v/v ethyl acetate/cyclohexane, retention factor of 0.36, revealer: phosphomolybdic acid) and evaporation.

Step 6: Esterification of the diol with an alkyl chain

The sixth and final step of the synthesis consists in esterifying the diol with two alkyl chains, so as to obtain the product A6. In this case, a C5 carbon-based chain was used.

The reaction is represented schematically as follows:

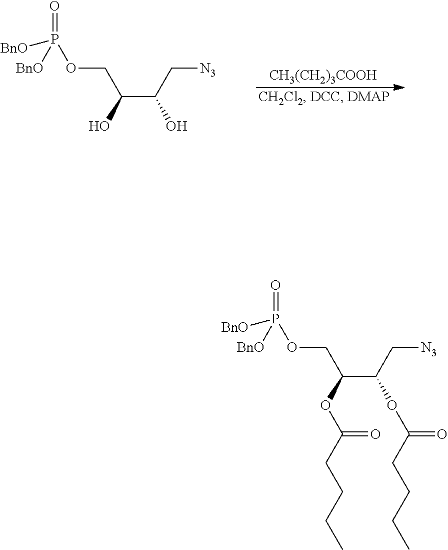

The product A5 (482 mg, 1.18 mmol), the valeric acid (362 mg, 3.55 mmol), the dicyclohexylcarbodiimide (732 mg, 3.55 mmol), the 4-dimethylaminopyridine (144 mg, 1.18 mmol) and finally the dichloromethane (12 ml) are added to a round-bottomed flask. This solution is stirred at ambient temperature for 15 h. After reaction, the reaction medium is filtered and washed with ethyl acetate. After filtration, the filtrate is concentrated by means of a rotary evaporator.

Finally, the product A6 (0.332 g, 0.58 mmol, 49% yield) is obtained in the form of a white solid after purification on a silica chromatography column (eluent: 1/9 v/v ethyl acetate/cyclohexane, retention factor of 0.65, revealer: phosphomolybdic acid) and evaporation.

Step B: Click Chemistry Reaction Between the m(PEG)₄₅-b-PPLG₁₉ Copolymer (Obtained in a Manner Similar to That Described in Example 1) and the Azide Derivative of Phosphatidic Acid, So as to Obtain a Copolymer of Formula (I) According to the Invention (Hereinafter “Protected Phospholipid Copolymer”)

This reaction is carried out in the presence of Cu(I)Br as catalyst and with PMDETA.

The reaction is represented schematically as follows:

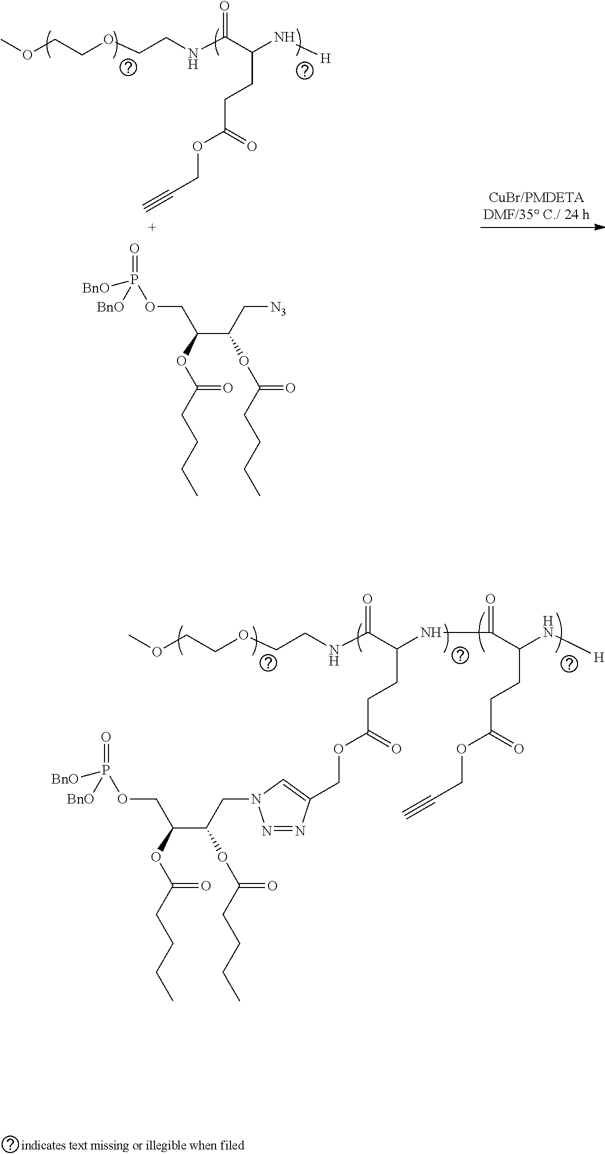

In a round-bottomed flask, 40 mg of copolymer (0.15 mmol of alkyne function) and 2 equivalents of the azide derivative of phosphatidic acid (165 mg, 0.29 mmol) are mixed into 5 ml of anhydrous DMF. The solution is degassed with argon for 30 minutes. A solution of 0.5 equivalent of Cu(I)Br (0.0108 g, 0.075 mmol) and of 1 equivalent of N,N,N′,N″,N″-pentamethyldiethylenetriamine (PMDETA, 31 μl, 0.15 mmol) in 5 ml of anhydrous DMF is degassed under argon for 30 minutes. The solution of Cu(I)Br and PMDETA is then added to the solution comprising the copolymer and the azide derivative of phosphatidic acid. The mixture is stirred for 24 h at 35° C. At the end of the reaction, the mixture is dialyzed for 2 days against a 10 mM EDTA solution and then for a further 4 days against milli-Q water. The solution is finally lyophilized in order to recover the final product.

### NMR Analysis

The spectra obtained are the following:

¹H NMR (DMSO, 300 MHz, δ ppm) : 7.36 (10H, 2*C₆H₅), 5.48 (2H, C—CH₂—O click), 5.21 (2H, N—CH₂), 5.03 (4H, 2*CH₂-C₆C₅), 4.60 (2H, O—CH₂—C non-click), 4.14 (4H, O—CH₂, N—CH₂), 3.50 (4H, —CH₂—CH_{2 PEG}), 2.28 (4H, 2*CH₂—CH₂—CH₂—CH₃), 1.46 (4H, 2*CH₂—CH₂—CH₂—CH₃), 1.24 (4H, 2*CH₂—CH₂—CH₂-—CH₃), 0.81 (6H, 2*CH₃).

### Step C: Deprotection of the Phosphate Group So as to Obtain a Copolymer of Formula (I) According to the Invention (Hereinafter “Deprotected Phospholipid Copolymer”)

The reaction is represented schematically as follows:

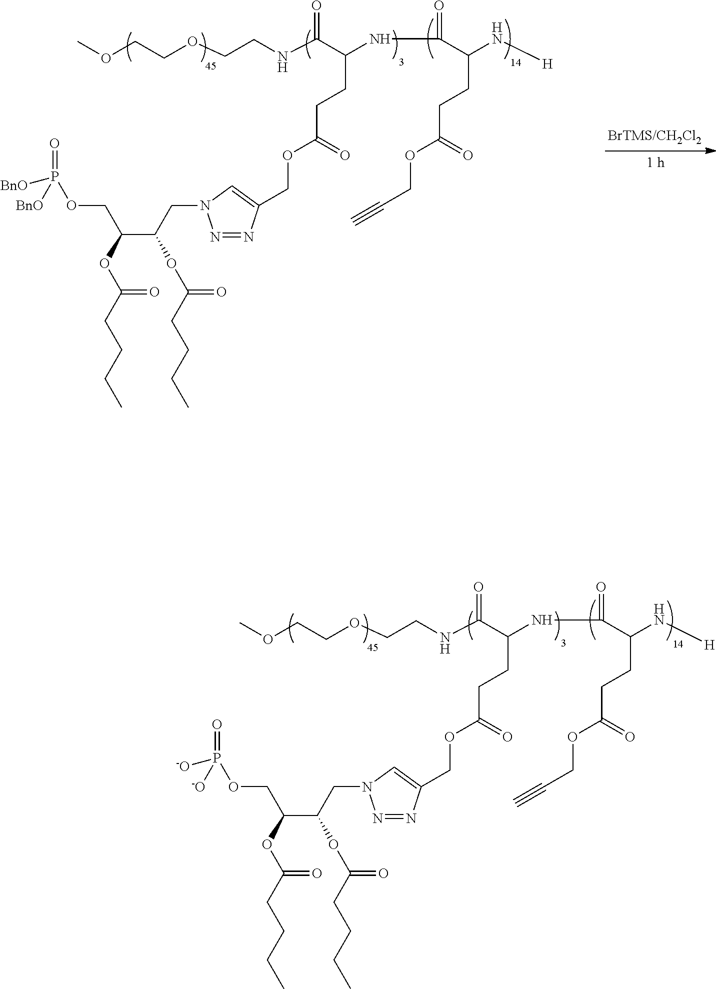

In a sample tube, 42 mg of the copolymer obtained in step B are dissolved in 1.6 ml of dichloromethane under an argon atmosphere, and the bromotrimethylsilane (0.350 ml, 2.6 mmol) is added. The reaction medium is then subjected to vigorous stirring for 1 h at ambient temperature. The solvent is then evaporated off under vacuum and the product obtained is taken up in 2 ml of methanol and subjected to vigorous stirring for 1 h at ambient temperature. The methanol is then evaporated off under vacuum. 5 cycles of washing with methanol/evaporation are then carried out in order to remove the impurities and to recover the deprotected phospholipid copolymer according to the invention.

### NMR Analysis

The spectra obtained are the following:

¹H NMR (DMSO, 300 MHz, δ ppm): 7.33 (10H, 2*C₆H₅), 5.49 (2H, C—CH₂—O click), 5.24 (2H, N—CH₂), 4.62 (2H, O—CH₂—C non-click), 3.50 (4H, —CH₂—CH_{2 PEG}), 2.28 (4H, 2*CH₂—CH₂—CH₂—CH₃), 1.46 (4H, 2*CH₂—CH₂—CH₂—CH₃), 1.27 (4H, 2*CH₂—CH₂—CH₂—CH₃), 0.84 (6H, 2*CH₃).

### Example 3

### Tests for Micellization of the Copolymers C4 and C9

### 1) Protocol

A solution of copolymers C4 and C9 is prepared by dissolving 10 mg of copolymer C4 or C9 in 2 ml of milli-Q water. The mixture is then vortexed for 5 minutes and, finally filtered using a 1 μm filter.

### 2) Results

The results show that the copolymer C4 is capable of forming micelles having an average diameter of between 15 nm and 120 nm. Virtually 12% of the micelles formed have an average diameter of 93 nm.

The copolymer C9 is capable of forming micelles having an average diameter of between 20 nm and 500 nm. Approximately 15% of the micelles formed have an average diameter of 197 nm.

### Example 4

### Pharmacokinetic Tests with Coagulation Factors as Therapeutic Proteins

The pharmacokinetic profiles of coagulation factors (FVII, FVIII or FIX) encapsulated in micelles of copolymer according to example 3 are determined after a single intravenous injection in catheterized OFA SD rats (bodyweight of 200-250 g) at 1 mg/kg, 100 or 200 IU/kg.

3 rats are injected with FVII-micelles, FVIII-micelles or FIX-micelles, 3 rats are injected with FVII, FVIII or FIX alone, and 2 control rats are injected with micelles alone in order to evaluate the toxicological effects.

The plasma is collected at several time points after injection (before injection, 5 min, 1 h, 3 h, 6 h, 24 h, 48 h, 72 h and 96 h). The blood samples are immediately treated on 10% citrate (sample: citrate ratio equal to 9:1; 3.13% by weight/volume), centrifuged at 1500 g for 15 minutes at 15° C., and stored at −20° C. before analysis.

The FVII, FVIII or FIX concentrations in the plasma are determined by ELISA, and the data are analyzed by non-compartmental analysis using the WinNonlin® software, version 6.3.

The pharmacokinetic parameters are determined. They include the maximum plasma concentration (Cmax), the area under the curve of the plasma concentration as a function of time starting from the time of administration of the dose until the final measurable concentration (AUCLast), the elimination half-life (t½), the distribution volume (Vd), the clearance (Cl) and the mean residence time (MRT).

1-19. (canceled)

20. A copolymer of formula (I) below: 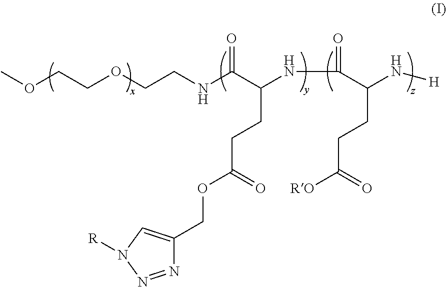
in which:
x is an integer between 10 and 250, preferably between 40 and 120,
y is an integer between 4 and 100, preferably between 10 and 100, preferably between 19 and 60,
z is an integer between 0 and (100−y), preferably equal to 0,
R represents an alkyl radical having from 1 to 10 carbon atoms, a phospholipid, a glycosaminoglycan, or an affinity ligand, and
R′ represents a hydrogen, the —CH₂—C≡CH group, a —CH₂-1H-1,2,3-triazole group, or a —CH₂—CH₂—CH₂—S—R″ group, where R″ represents an alkyl radical having from 1 to 10 carbon atoms, a phospholipid, a glycosaminoglycan, an affinity ligand or an imaging probe.

21. The copolymer as claimed in claim 20, wherein the alkyl radical having from 1 to 10 carbon atoms is chosen from methyl, ethyl, n-propyl, isopropyl, n-butyl, sec-butyl, isobutyl, tert-butyl, n-pentyl, isopentyl, neopentyl, n-hexyl, n-nonyl, 2-methylcyclopentyl and 1-cyclohexylethyl radicals.

22. The copolymer as claimed in claim 20, wherein R represents an alkyl radical having from 4 to 9 carbon atoms, a phospholipid or a glycosaminoglycan and/or in that R′ represents a hydrogen or the —CH₂—CCH group.

23. The copolymer as claimed in claim 20, wherein the phospholipid is chosen from phosphoglycerides and sphingomyelins.

24. The copolymer as claimed in claim 20, wherein the glycosaminoglycan is heparin.

25. The copolymer as claimed in claim 24, wherein the heparin is chosen from natural heparin, enoxaparin, tinzaparin, nadroparin and fondaparinux.

26. The copolymer as claimed in claim 20, wherein R is phosphatidic acid, preferably phosphatidic acid formed by esterification of two fatty acids, which are preferably saturated, having a carbon-based chain of from 3 to 22 carbon atoms, and of a phosphoric acid with a glycerol.

27. The copolymer as claimed in claim 20, wherein R is the phosphatidic acid of formula below: 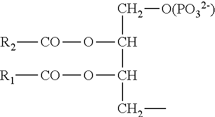
where R₁ and R₂ are identical and represent an aliphatic chain of a fatty acid having from 3 to 22 carbon atoms, preferably R₁ and R₂ are each —(CH₂)₂—CH₃ or —(CH₂)₃—CH₃.

28. The copolymer as claimed in claim 20, wherein R is identical or different among the triazole glutamate-derived monomers.

29. A pharmaceutical composition comprising, in a pharmaceutically acceptable medium, at least one copolymer as defined in claim 20, and at least one therapeutic protein.

30. The pharmaceutical composition as claimed in claim 29, wherein the therapeutic proteins are chosen from antibodies, the coagulation factors I, II, V, VII, VIIa, VIII, IX, X, XI, XII or XIII, or Willebrand factor, modified coagulation factors, factor H, ITI, alpha 1-antitrypsin, antithrombin, albumin, fibrinogen, human prothrombin complex, protein C, insulin and erythropoietins.

31. A method comprising the step of encapsulating of one or more proteins with a copolymer as defined in claim 20.

32. The method as claimed in claim 31, wherein said one or more proteins are therapeutic proteins.

33. A process for preparing a copolymer as defined in claim 20, comprising:
a) a step of Click chemistry between the alkyne of formula (II) below: 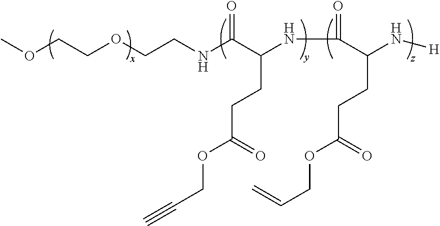
in which:
x is an integer between 10 and 250, preferably between 40 and 120,
y is an integer between 4 and 100, preferably between 10 and 100, preferably between 19 and 60, and
z is an integer between 0 and (100−y), preferably equal to 0,
and a compound of formula R—N₃, R being as defined in o claim 20, in the presence of copper(I), in order to obtain the compound of formula (III): 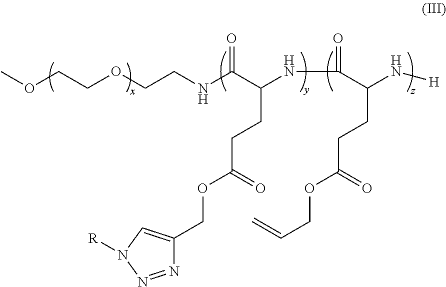
then
b) preferably, when z is other than 0, a step of modifying the allyl function of the compound of formula (III).

34. The process as claimed in claim 33, wherein the Click chemistry step is carried out in the presence of Cu(I)Br, PMDETA and DMF, at a temperature of between 30° C. and 40° C., and for a time of between 20 and 40 hours.

35. The process as claimed in claim 33, wherein step b) is a step of deprotecting the allyl protective group of the compound of formula (III), preferably by oxidation and cleavage of the allyl protective group, in order to obtain a copolymer of formula (I) in which R′ is a hydrogen.

36. The process as claimed in claim 33, wherein step b) is a step of Click chemistry between the compound of formula (III) and a thiol of formula R″—SH, where R″ represents an alkyl radical having from 1 to 10 carbon atoms, a phospholipid, a glycosaminoglycan, an affinity ligand or an imaging probe, in order to obtain a copolymer of formula (I) in which R′ is a —CH₂—CH₂—CH₂—S—R″ group.

37. The process as claimed in claim 33, wherein, when z is equal to 0 for the compound of formula (II), step a) of Click chemistry between the alkyne of formula (II) and the compound of formula R-N₃ is carried out incompletely, so as to obtain a copolymer of formula (I): 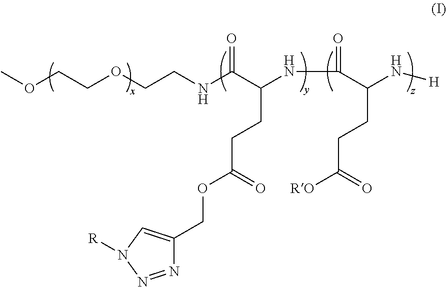
in which R′ represents the —CH₂—CCH group.

38. Micelles obtained from copolymers as defined in claim 20, and comprising an encapsulated therapeutic protein.

39. A pharmaceutical composition comprising the micelles as defined in claim 38.
